(19) United States
(12) Patent Application Publication
(10) Pub. No.: US 2015/0196359 A1
(43) Pub. Date: Jul. 16, 2015

(54) METHODS FOR DELIVERY OF SUB-SURFACE ARRAY OF ABSORBER MATERIALS AND METHODS OF LIGHT IRRADIATION THERAPY

(71) Applicant: Sebacia, Inc., Duluth, GA (US)

(72) Inventor: Dilip Paithankar, Wayland, MA (US)

(21) Appl. No.: 14/593,758

(22) Filed: Jan. 9, 2015

Related U.S. Application Data
(60) Provisional application No. 61/926,097, filed on Jan. 10, 2014.

(51) Int. Cl. A61B 18/20; A61M 37/00

(52) U.S. Cl. CPC A61B 18/20; A61M 37/0015; A61M 2037/0061

(57) ABSTRACT

Methods for injecting light absorbing materials or particles into the skin and irradiating the materials or particles to create zones of thermal injury are provided. Such methods can be used to treat the effects of aging and/or photoaging, among other indication.

### CROSS REFERENCE TO RELATED APPLICATION

This application claims the benefit or priority under 35 U.S.C. §119(e) of U.S. Provisional Application Nos. 61/926,097 filed Jan. 10, 2014, the entire contents of which are incorporated herein by this reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to methods for delivery of sub-surface arrays of absorber materials and methods of light irradiation therapy.

### BACKGROUND

Photoaging is aging of skin with exposure to ultraviolet (UV) light. UV exposure can cause appearance of wrinkles. Also, with aging, skin can become lax. Both conditions can be improved by remodeling of the collagen to decrease the appearance of wrinkles. Successful treatments including demabrasion, chemical peels, and CO2 or Er:YAG laser resurfacing have been developed with excellent results. However, the side effects of oozing, bleeding, crusting, erythema, etc. as a healing response to the contiguous bulk thermal injury make such therapies less attractive. The second generation technology involved cooling of the surface and performing sub-surface thermal injury. Here, results were inadequate when the side effects profile and pain were kept to minimum. The third generation technology was the fractional treatment of skin wherein plugs of skin were removed or heated via ablative or non-ablative laser or mechanical sharp hollow needles. This minimizes side effects with acceptable results. However, the best results of fractional treatments are noted when high coverage is seen but the side effects can become problematic.

While certain approaches have been attempted, improvements are still needed in the field of treatment for lax skin and wrinkles.

### SUMMARY OF THE DISCLOSURE

The present invention relates to a method of treating at least one of aging and photoaging effects on skin. The method comprises injecting an amount of light absorbing material at a target zone beneath the skin in a predetermined pattern at a predetermined depth; and irradiating the light absorbing material to selected wavelengths of light, thereby causing at least one zone of thermal injury. Injecting can comprise injecting using a single needle or a microneedle array. When using a microneedle array, injecting can comprise depressing a reservoir in fluid connection with at least one of the needles in the array. The depressing can be performed manually or using a delivery device. The predetermined pattern can comprise regularly spaced rows and columns or the pattern can be irregular. The light absorbing material can be configured to absorb light at a wavelength of about 800 nm to about 1,200 nm. Irradiating the light absorbing material can comprise exposing the light absorbing material to light with a wavelength band of about 700 to about 1,200 nm. Injecting an amount of light absorbing material can comprise injecting at a depth of about 50 microns to about 2 mm. A diameter of the thermal damage zone can be about 50 microns to about 1 mm. Irradiating the light absorbing material can cause a plurality of zones of thermal injury, and a density of the thermal damage zones can be about 10 per cm² to about 15,000 per cm². An absorption coefficient in the target zone can be about 1.0 to about 1,000 (l/cm).

In another aspect, a device for treating skin is provided. The device comprises an array of microneedles; and a reservoir configured to hold fluid comprising a light absorbing material fluidly connected to at least one of the microneedles. The array can comprise microneedles in regular rows and columns or in an irregular pattern. The device can be contoured to match to a treatment site of a patient. The device can be a platform comprising a large microneedle array or a plurality of microneedle arrays. The device can comprise at least two reservoirs, each reservoir fluidly connected to at least one microneedle. The device can comprise a rectangular or ovular shape.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of treating skin by injecting an array of light absorbing material.

FIGS. 2A-2C illustrate embodiments of various patterns of injection.

FIGS. 3A-3C illustrate various views of an embodiment of a microneedle array applicator.

FIGS. 4A-4E illustrate various embodiments of differently shaped applicators comprising microneedle arrays.

FIG. 5 illustrates an embodiment of a microneedle array applicator comprising single reservoir.

FIG. 6 illustrates an embodiment of a microneedle array applicator comprising a plurality of reservoirs.

FIG. 7 illustrates embodiments of contoured microneedle arrays.

FIG. 8 illustrates an embodiment of a mask applicator comprising a microneedle array or arrays.

FIG. 9 illustrates an embodiment of an entire face mask applicator comprising a microneedle array or arrays.

FIG. 10 illustrates an embodiment of a delivery device that can be used during injection.

### DETAILED DESCRIPTION

One area of development in aging skin treatment includes methods for attempting to provide levels of more intense injury but in a sub-surface location in a fractional mode while while leaving the top skin viable. One particular approach is with ultrasound. More specifically, there have been attempts with focused ultrasound (e.g., Ulthera). However, the dimensions of the zones of thermal injury are limited by the wavelength of the ultrasound waves. There still remains a need for more precise, but intense, injury to sub-surface target sites without the inherent limitations of the methods described above.

In one aspect of the present invention, control over the amount and spread of the sub-surface thermal lesion can be obtained by injecting, in an array pattern, doses of a suspension of microparticles underneath the skin. In one aspect of the present invention, injecting in an array pattern, in the microliter range of suspension, underneath the skin in the region for therapy can be performed. The amount of suspension per dose can be controlled by the amount of injection or by the size of the delivery device. In addition, the depth and the lesion-to-lesion distance may also be varied based on patient need or desired therapeutic outcome. The concentration of particles in the dose, amount of suspension per dose, the depth of injection, and/or the lesion to lesion distance can be adjusted to effect a desired treatment outcome.

In some embodiments of the present invention, a composition of microparticle chromophores (light energy adsorbing materials) are injected into the sub-surface of the skin of a person needing or desiring treatment. In a preferred embodiment, the microparticle chromophores composition comprises unassembled plasmonic nanoparticles. Such plasmonic nanoparticles might include nanorods, hollow nanoshells, silicon nanoshells, nanoplates, nanorice, nanowires, nanopyramids, nanoprisms, nanoplates nanoplates, solid nanoshells, hollow nanoshells, nanorods, nanorice, nanospheres, nanofibers, nanowires, nanopyramids, nanoprisms, nanostars and other configurations known to those skilled in the art. The plasmonic nanoparticles are generally of a size from 1 to 1000 nm, although it is preferred that the plasmonic nanoparticles are of a size between about 100 and 300 nm. The plasmonic nanoparticles typically comprise silver, gold, nickel, copper, titanium, silicon, galadium, palladium, platinum, or chromium. Such plasmonic nanoparticles generally have a peak adsorption in the near infra-red region of the electromagnetic spectrum.

In a further preferred embodiment, the injected composition comprises plasmonic nanoparticles such as gold nanoshells having a silica core and a gold shell (diameter 150 nm). In a still further preferred embodiment, nanoshells used are composed of a 120 nm diameter silica core with a 15 micron thick gold shell, giving a total diameter of 150 nm. Such nanoshells may be covered by a 5,000 MW PEG layer. The PEG layer prevents and/or reduces nanoshell aggregation, thereby increasing the nanoshell suspensions stability and shelf-life.

The composition comprising plasmonic nanoparticles may have a nanoparticle concentration in a range of about 10⁹ to about 10¹⁶ nanoparticles per ml. Typically, the composition comprising plasmonic nanoparticles may have a nanoparticle concentration in a range of about 10¹⁰ to about 10¹³ nanoparticles per ml. Such nanoparticle concentrations are calculated from the optical density of the composition at its peak absorption wavelength. Compositions of plasmonic nanoparticles used in the present invention will generally have an optical density between about 0.05 and about 5000.

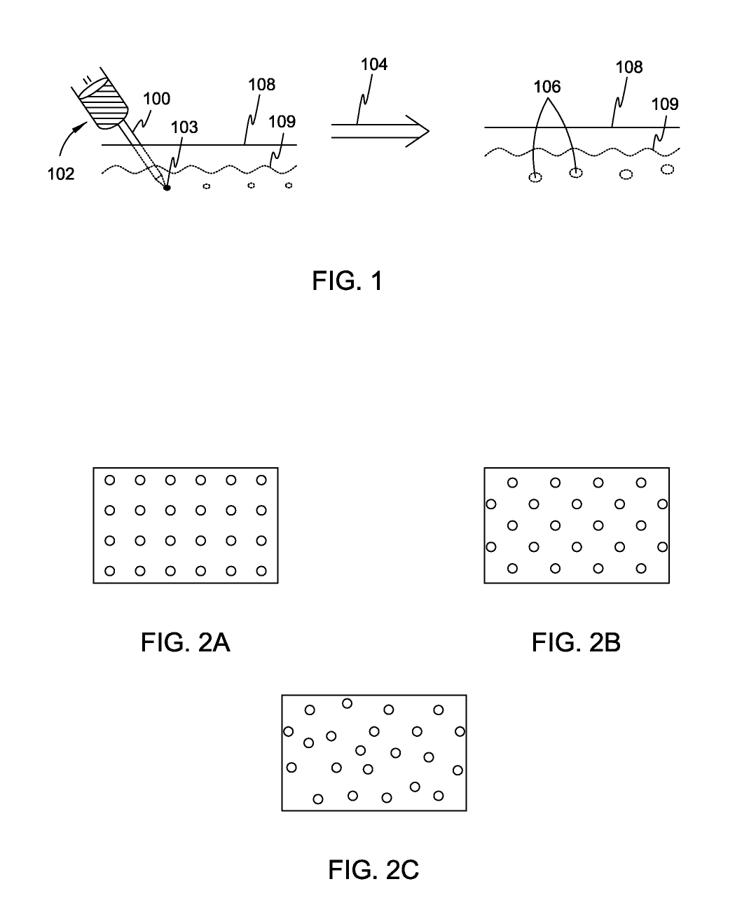

In some embodiments, an individual needle or syringe can be used to produce array patterns. FIG. 1 illustrates a side cross-sectional view of a needle 100 injecting a light absorbing material 102 at point 103 (in an array pattern) below the surface of both the epidermis 108 and the dermis 109. FIG. 1 illustrates a laser 104 (the wavelength emitted by laser 104 being in the near-IR range, such as 810 nm) being applied to the injected light absorbers 102. When light absorbers 102 are plasmonic nanoparticles, once these particles absorb the near-IR light, they heat their local environment and produce thermal damage zones 106 at or near the locations of the injected light absorbers 102. As shown in FIG. 1, the light absorber 102 is being injected below the epidermis 108, into the dermis.

FIGS. 2A-2C illustrate top views of embodiments of injection patterns. The regions of injection of FIG. 2A form a regular pattern, with rows and columns. FIG. 2B illustrates an embodiment of a pattern of injection in which the regions of injections form offset or staggered rows and columns. FIG. 2C illustrates an embodiment of an injection pattern that is irregular. For example, the pattern may be suited to an individual patient need or desired treatment outcome.

In one aspect, very small amounts are needed to get the desired effect. Typical amount may be 10 nl per injection when the absorption coefficient is 5 cm⁻¹. The range can be about 0.1 to 1,000 nl or about 1 to 100 nl. The absorption coefficient can be about 1 cm⁻¹ to 1,000 cm⁻¹. In one particular aspect, there is provided light absorbing materials in the near infrared (IR) range, e.g., about 700 to about 1,200 nm. For example, the light absorbing material can be configured to absorb at about 755 nm, about 800 nm, about 810 nm, or about 1,064 nm. Such materials can be used with intense pulsed light instruments (IPLs) with a wavelength band of about 700 to about 1,200 nm. Where the light absorbing materials are plasmonic gold nanoshells, the laser may be tuned to the nanoshell's absorption peak (40-50 J/cm², 30-ms, 9×9 mm, LightSheer (800 nm)).

It is preferred that the thermal injury produced by the process of the present invention is substantially confined to a region, an “island,” surrounding the injected light absorbing material. In certain preferred embodiments of the present invention, the duration of the laser pulse is the same as, or less than, the thermal relaxation time of the zone of the light absorbing material. It is preferred that the duration of the laser pulse is between about 0.1 ms and about 200 ms. Such pulses are believed to produce the desired islands of injury.

The absorption coefficient in the target zone can be in the range of about 1.0 to about 1,000 (l/cm). The depth of the injections and subsequent thermal lesions can be about 50 microns to about 2 mm. The depth can depend on the anatomical site of treatment and/or a skin thickness of the patient. A diameter of the thermal damage zone can range from about 50 microns to about 1 mm. The diameter of the zone can depend on the amount of suspension injected beneath the skin. The density of the thermal damage zone can range from about 10 per cm² to about 15,000 per cm². The density of thermal damage ones can depend on the diameter of the zone, or the amount of suspension injected.

In addition to individual injection of the particle pattern, an array of microneedles can be used to inject the material to the desired depth within the skin. In some embodiments, the microneedle array particle delivery device is a patch based reservoir and needle array that may be provided in any of a wide variety of sizes, shapes and configurations.

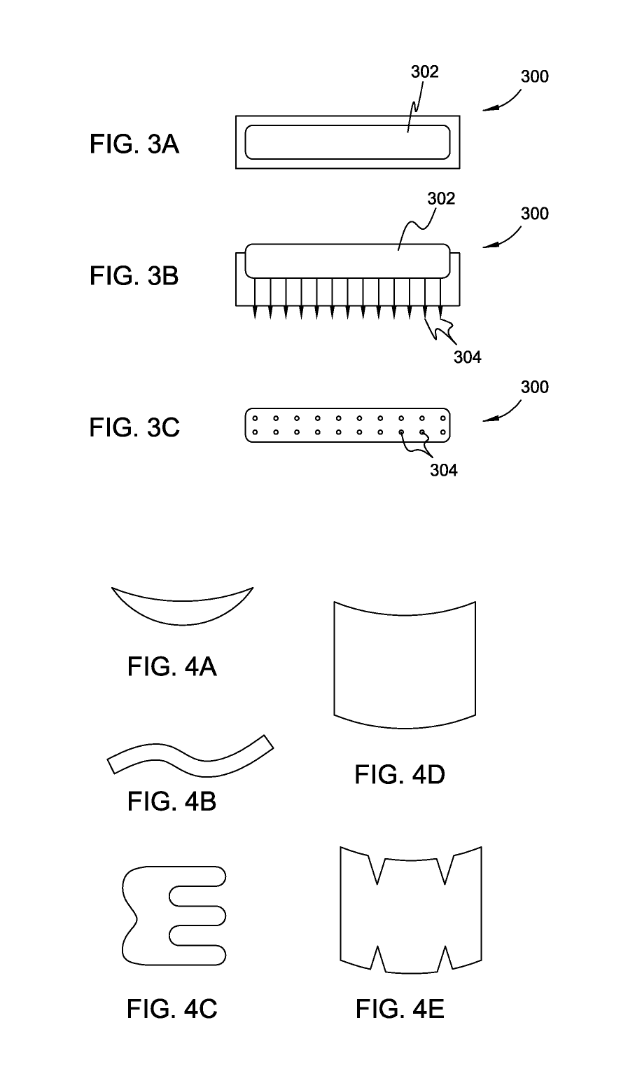

FIG. 3A illustrates a top view of an embodiment of a microneedle array applicator 300 including a reservoir 302 of light absorbing particles for therapy (e.g., provided in a suspension). FIG. 3B illustrates a side view of the microneedle array applicator 300 in which the individual needles 304 are visible. FIG. 3C illustrates a bottom view of the applicator which the pattern of the needles 304 is shown. In this embodiment, the microneedle pattern comprises regularly spaced rows and columns.

As noted above, in some embodiments, the pattern comprises irregularly spaced rows and columns. The pattern of injection can comprise a generally irregular pattern (e.g., based on individual patient characteristics or desired treatment outcome). The pattern of injection can be shaped in other ways as well. For example, the pattern may be circular, rectangular, ovular, etc. The pattern can be selected based on patient anatomy and/or desired treatment outcome.

The needles 304 can be sized so that they are all a same length and gauge. In some embodiments, the needles 304 have varying lengths and/or gauges. The needles can be between about 50 microns and about 2 mm long. The relative locations of needles with different depths and/or gauges can be selected based on the anatomy, the anatomy of a particular patient, and/or a desired treatment outcome.

The microneedle array 300 shown in FIGS. 3A-3C is rectangular in shape, with rounded edges; however, other shapes are also possible. FIGS. 4A-4E illustrate top views of embodiments of non-rectangular shaped microneedle arrays. The shape of the arrays can be selected based on the particular anatomy to be treated. For example, if the patient is to be treated in particularly discrete, a smaller or thinner array may be used. A skilled artisan will appreciate that other shapes are also possible.

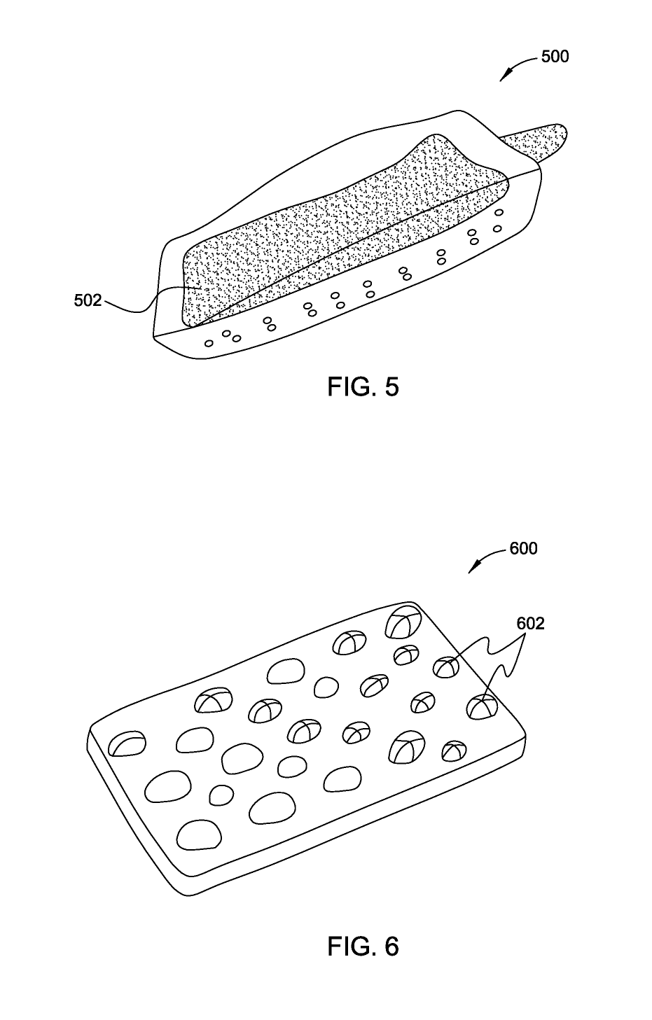

FIG. 5 illustrates a microneedle array applicator 500 comprising a single reservoir 502 of light absorbing materials, similar to that shown in FIGS. 3A-3C. Such a microneedle array applicator 500 may be suitable for general area dispersion, when it is desired to inject materials over a large area. The underside (not shown) of applicator 500 has a microneedle array selected for desired pattern and depth of sub-dermal injection of light absorbing materials.

FIG. 6 illustrates a microneedle array applicator 600 comprising a plurality of reservoirs 602 of light absorbing material. Such an applicator 600 can allow for more specific, localized treatment using one or a group of needles in a particular area or areas. Each reservoir 602 can be associated with a single needle or a group of adjacent needles. The reservoirs 602 can be of different sizes, but are the same size in some embodiments. Each reservoir 602 can include the same light absorbing material or different light absorbing materials. The underside of applicator 600 (not shown) has a microneedle or an array array of microneedles associated with each reservoir 602 selected for desired pattern and depth of sub-dermal injection of light absorbing materials.

As described in FIGS. 4A-4E, microneedle particle delivery arrays may have a specific shape tailored to the treatment site. Contour of the body or injection site may provide the guide for the shape, size, or other characteristic of the microneedle particle injector. Thus, microneedle arrays comprising a contoured shape may be utilized in some embodiments.

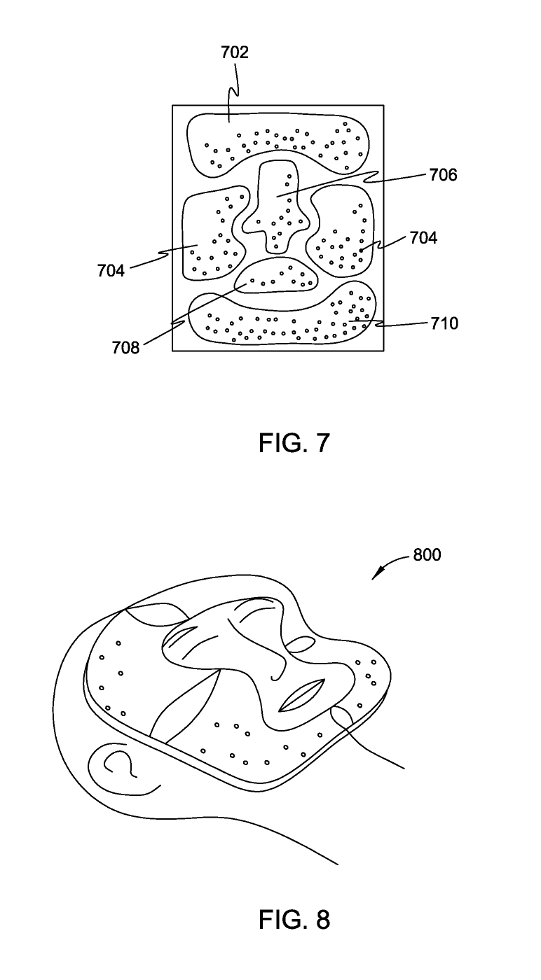

FIG. 7 illustrates an example of a contoured, custom shaped microneedle array for treatment. In some embodiments, a microneedle array pad 702, shaped to contour to a forehead of a patient, contains a reservoir of light absorbing material for delivery via an associated micro-needle array. Microneedle array pads 704, shaped to contour to a cheek or cheeks of a patient, contains a reservoir of light absorbing material for delivery via an associated micro-needle array. A microneedle array pad 706, shaped to contour to a nose of a patient, contains a reservoir of light absorbing material for delivery via an associated micro-needle array. A microneedle array pad 708, shaped to contour to a mouth of a patient, contains a reservoir of light absorbing material for delivery via an associated micro-needle array. A microneedle array pad 710, shaped to contour to a chin or jawline of a patient, contains a reservoir of light absorbing material for delivery via an associated micro-needle array. The size and shape of the contoured arrays or overall platform can be precisely matched to a patient using scans or measurements of the patient treatment site. While FIG. 7 only depicts arrays shaped for the face of a patient, arrays shaped for treatment of other areas, such as the hands or neck, are also possible.

In some embodiments, more than one microneedle array can be used at a time. FIG. 8 illustrates an embodiment of a custom shaped mask 800 that can comprise a plurality of arrays or a large array of microneedles. The mask 800 can be articulated to allow for positioning and application. The mask 800 can include a single reservoir, or multiple reservoirs, of a composition of light absorbing materials. The reservoir configuration can be selected based on the anatomy or desired treatment outcome. The underside of mask 800 (not shown) can include similar or differently configured microneedles as described herein. While a mask is shown in FIG. 8, platforms for treatments of other areas are also possible. For example, a platform comprising a large microneedle array or multiple arrays can be shaped to contour to and configured to treat the neck or chest area.

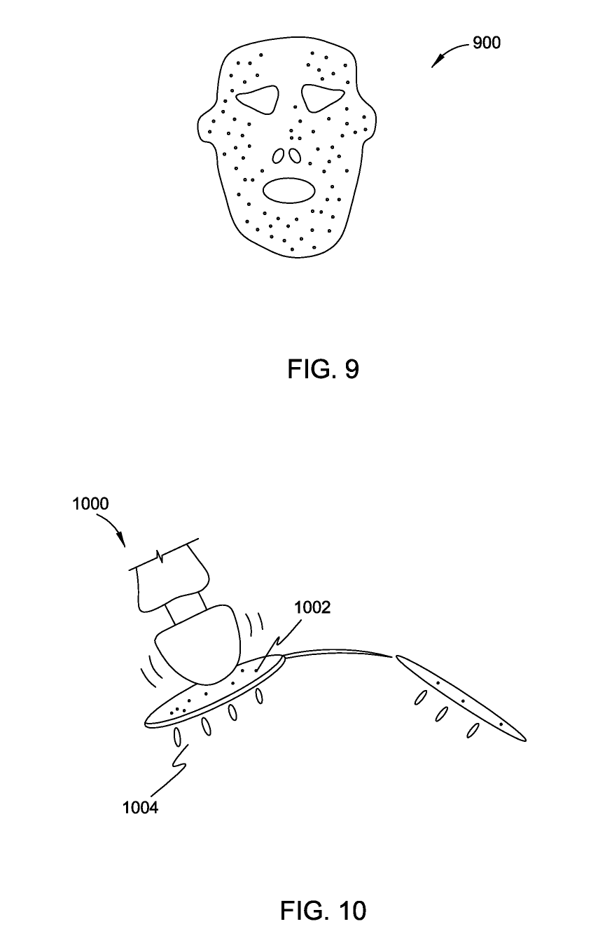

FIG. 9 also illustrates an embodiment of a mask 900, the underside of which (not shown) can comprise a large array of microneedles, or a plurality of arrays of microneedles. The selected treatment area can be scanned and a custom fit microneedle array or arrays and delivery reservoir or reservoirs can be provided in mask 900.

In some embodiments, the reservoirs are pressed manually to release the light absorbing materials. In some embodiments, a delivery device can be used. FIG. 10 illustrates an embodiment of a delivery device 1000 that can be used to depress the particle reservoir 1002 of a microneedle array 1004. The tool may be useful in fully depressing the reservoir or the reservoir may be depressed manually. Control or precision in the amount of particle material injected into a part of the needle array may be controlled by the size of the reservoir. When fully depressed or flattened out, the user knows that the entire contents of the reservoir have been delivered. The reservoir size is selected based on the dose to be delivered. Whether to a large area (FIG. 5) or to a smaller area (FIG. 6) the size and delivery technique may be adjusted to fit the circumstances of a particular needle array or treatment pattern.

The device can provide a way to adjust the volume of the injected material in the needle set. For example, a delivery device including a trigger can be used to depress a reservoir. A squeeze of a trigger can advance a plunger in the delivery device by a known distance that is controllable, thus adjusting the volume injected.

### Alternative Indications for Treatment

Similarly, light absorbing particles can be injected into sweat glands and then the skin can be treated with light to thermally damage and inactivate the glands to treat hyperhidrosis. The damage profile can be controlled via control of density, depth, and amount per injection.

In one aspect, the skin in the treatment area has been damaged by burns or includes a skin graft used to repair burned skin. The specific injection patterns, materials and density described herein adapted and configured to promote healing of burn skin or adaptation to skin graft or grafted region.

A variety of different formulations and compositions may be used to provide the activatable particles for the uses described herein. The examples and illustrations included herein show, by way of illustration and not of limitation, specific embodiments in which the subject matter may be practiced. In another aspect, the operation of the delivery device (i.e., delivery by injection of particles by any suitable means) for the delivery of a delivery fluid precedes or follows another treatment or another desired therapy. In this case, the operation and use of the delivery device is one part of a multi-part therapy. In one specific example of a multiple part therapy is the use of the delivery system to deliver a fluid, a formulation particles, shells, pharmaceuticals, liposomes, other treatment agents or pharmacologic materials onto, into or within a structure within a treatment or delivery site followed by a further treatment of the delivery or treatment site. In addition to the examples above, one specific example the further treatment is providing an activating energy to a fluid, a formulation or a pharmacologic material. Exemplary fluids, formulations and treatments are described in U.S. Pat. No. 6,183,773; U.S. Pat. No. 6,530,944; U.S. Published Patent Application US 2013/0315999 and U.S. Published Patent Application US 2012/0059307, each of which is incorporated herein in its entirety. Additionally or optionally, one or more of the delivery device operating parameters, and/or methods of use of the delivery system described herein may be modified based upon one or more characteristics of the delivery fluid, a component of the delivery fluid or a particle within the delivery fluid being used. In some embodiments, the particle being delivered may include one or more of, for example, nanorods, nanoshells, nanoprisms, dyes such as rose Bengal, ICG, methylene blue.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for treating skin comprising
injecting an amount of light absorbing material at a target zone beneath the skin in a predetermined pattern at a predetermined depth; and
irradiating the light absorbing material to selected wavelengths of light, thereby causing at least one zone of thermal injury.

2. The method of claim 1, wherein the injecting comprises injecting using a single needle.

3. The method of claim 1, wherein the injecting comprises injecting using a microneedle array.

4. The method of claim 3, wherein injecting the light absorbing material comprises depressing a reservoir in fluid connection with at least one of the needles in the microneedle array.

5. The method of claim 4, wherein depressing the reservoir can be performed manually or using a delivery device.

6. The method of claim 1, wherein the predetermined pattern comprises regularly spaced rows and columns.

7. The method of claim 1, wherein the predetermined pattern is irregular.

8. The method of claim 1, wherein the light absorbing material is configured to absorb light at a wavelength of about 800 nm to about 1,200 nm.

9. The method of claim 1, wherein irradiating the light absorbing material comprises exposing the light absorbing material to light with a wavelength band of about 700 to about 1,200 nm.

10. The method of claim 1, wherein injecting an amount of light absorbing material comprises injecting at a depth of about 50 microns to about 2 mm.

11. The method of claim 1, wherein a diameter of the thermal damage zone is about 50 microns to about 1 mm.

12. The method of claim 1, wherein irradiating the light absorbing material causes a plurality of zones of thermal injury, and wherein a density of the thermal damage zones is about 10 per cm2 to about 15,000 per cm2.

13. The method of claim 1, wherein an absorption coefficient in the target zone is about 1.0 to about 1,000 (l/cm).

14. A device for treating skin, comprising
an array of microneedles; and
a reservoir configured to hold fluid comprising a light absorbing material fluidly connected to at least one of the microneedles.

15. The device of claim 14, wherein the array comprises microneedles in regular rows and columns.

16. The device of claim 14, wherein the array comprises microneedles arranged in an irregular pattern.

17. The device of claim 14, wherein the device is contoured to match to a treatment site of a patient.

18. The device of claim 14, wherein the device is a platform comprising a large microneedle array or a plurality of microneedle arrays.

19. The device of claim 14, wherein the device comprises at least two reservoirs, each reservoir fluidly connected to at least one microneedle.

20. The device of claim 14, wherein the device comprises a rectangular or ovular shape.

21. The method of claim 9, wherein the pulse duration of the laser matches or is lower than the thermal relaxation time of the zone of the light absorbing material and is in the range of 0.1 ms to 200 ms.
